(19) United States
(12) Patent Application Publication
(10) Pub. No.: US 2005/0153948 A1
(43) Pub. Date: Jul. 14, 2005

(54) Methods and formulations for the treatment of medical conditions related to elevated dihydrotestosterone

(75) Inventor: Curtis A. Spilburg, Chesterfield, MO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Zomanex, LLC, Chesterfield, MO (US)

(21) Appl. No.: 10/989,173

(22) Filed: Nov. 15, 2004

Related U.S. Application Data
(60) Provisional application No. 60/523,613, filed on Nov. 20, 2003.

(51) Int. Cl. A61K 31/56

(52) U.S. Cl. 514/171

(57) ABSTRACT

The present invention describes a composition that contains a plant sterol or plant stanol or their fatty acid esters and an emulsifier for treating conditions that are related to elevated dihydrotestosterone. The compositions can be prepared in a dry form for use as a food ingredient, tablet or capsule. Alternatively, the compositions can be dissolved in oil.

### CROSS-REFERENCE

This application claims the benefit of U.S. Provisional Application No. 60/523,613, filed Nov. 20, 2003 which is incorporated by reference in its entirety.

### BACKGROUND OF THE INVENTION

This invention relates generally to the use of natural products like soy sterols and soy lecithin in a method for the treatment and improvement of the symptoms associated with elevated dihydrotestosterone, such as benign prostatic hyperplasia (BPH).

Benign prostatic hyperplasia (BPH) refers to the enlargement of the prostate gland, which occurs in 50% of 60-year old men with its incidence increasing with age to 90% at 85 years [Berry, S J, Coffey, D S, Walsh, P C, et al., The development of human benign prostatic hyperplasia with age. J. Urol. 132: 474-479, 1984]. This common age-related condition causes both obstructive and irritative lower urinary tract symptoms, characterized by dysuria, frequency, nocturia, sense of incomplete emptying and others. With such a high rate of incidence, BPH is the cause of considerable morbidity and health care costs and, with an aging population, it is expected that it will produce even more hospitalizations than the current 380,000 per year. Indeed, surgical treatment of BPH is the second most common procedure in the Medicare population with 25% of American males treated by age 80 [Barry, M J, Fowler, F J, O'Leary, M P, et al., The American Urological Association symptom index for benign prostatic hyperplasia. J. Urol. 148: 1549-1557, 1992].

The cause of prostate enlargement has not been established, but accumulated evidence from animal and human studies suggests that development of BPH is mediated by an imbalance of cell proliferation and cell death. To explain the genesis and subsequent course of the disease, it has been hypothesized that proliferative processes within the prostate are enhanced while apoptotic processes are inhibited, to produce an increase in cell number and a subsequent enlargement of the gland. Since sex hormones play a pivotal role in the development and growth of the normal prostate, their role in the progression of BPH has come under careful scrutiny.

For the maintenance of prostate health and homeostasis, the androgens testosterone and its reduced form, dihydrotestosterone (DHT), are of critical importance. The testicular Leydig cells synthesize over 95% of testosterone to produce an average concentration in adult male plasma of about 22 nmol/L. Free testosterone diffuses into the prostate cell where it is converted irreversibly to DHT by the NADPH-dependent enzyme 5α-reductase. The reductase has two isoenzymes, one located on chromosome 5 (Type I) and the other on chromosome 2 (Type II), but it is believed that in the human prostate the Type II enzyme is the predominant form [Russell, D W, Wilson, J D. Steroid 5α-reductase: two genes/two enzymes. Ann. Rev. Biochem. 63: 25-61, 1994]. As a result of the enzymatic activity of the Type II 5α-reductase, the concentration of DHT in the prostate is about five times higher than that of testosterone, while in serum the DHT concentration is about 5-10-fold less than that of testosterone.

While both sex hormones are readily accessible to mediate physiological processes, evidence strongly suggests that in fact DHT is the likely agent for the differentiation of the fetal prostate and development of male genitilia. Moreover, evidence indicates that this same androgen is also the primary causative agent in the development of BPH. For example, the incidence of BPH symptoms coincides with those decades in life when circulating levels of both total and free testosterone are decreasing. In contrast, over this same age span, DHT concentrations do not decrease appreciably, indicating that DHT is the hyper-plastic agent, not testosterone.

A number of biochemical experiments provide a molecular framework for understanding the various cellular events that are mediated preferentially by DHT. DHT interacts with the androgen receptor, a member of the nuclear receptor superfamily, with greater binding affinity than that for testosterone [Griffiths, K., Morton, M S, and Nicholson, R I: Androgens, androgen receptors, antiandrogens and the treatment of prostate cancer. Eur Urol 32 (suppl 3): 24-40, 1997]. Based on this preferential binding affinity, it is likely that DHT is responsible for most of the androgen-based physiological effects found in the prostate gland. Once DHT binds to the androgen receptor localized on the nuclear membrane, the receptor undergoes a conformational change that allows it to bind to DNA, which in turn produces mRNA specific for a number of growth factors, regulatory proteins and other signaling factors. [Marcelli M, and Cunningham G R, Hormonal signaling in prostatic hyperplasia and neoplasia. J. Clin Endocrinol 126: 1165-1172, 1999; Griffiths, K: Molecular control of prostate growth, in Kirby, R, McConnell, J D, Fitzpatrick, J, et al (Eds), Textbook of Benign Prostatic Hyperplasia. Oxford, UK, Isis Medical Media Ltd., 1996, pp 23-26]. It is the intricate interplay between these DHT-induced proteins that provides the potential for prostatic hyperplasia. For example, DHT not only enhances cell proliferation by controlling the expression of epidermal growth factor and keratinocyte growth factor, but it also modulates the activity of transforming growth factor, a protein that is known to modulate apoptosis [Griffiths, see above; Kim, I Y, Zelner, D J, Sensibar, J A, et al., Modulation of sensitivity to transforming growth factor-beta 1 and the level of type II TGF-β receptor in LNCaP cells by dihydrotestosterone. Exp. Cell Res. 222: 103-110, 1996].

The medical management of BPH includes surgical therapies such as transurethral prostatic resection (TURP), open prostatectomy and transurethral needle ablation and non-invasive pharmacological approaches that are directed at biochemical pathways. While the surgical procedures are used to treat extreme cases of BPH their use is complicated by a re-treatment rate of 20% after 8 years with an overall incidence of complications of 16%. Because of the recent advances in the understanding of the biochemical basis of BPH, pharmaceutical approaches provide an attractive treatment for BPH treatment.

With the recognition that DHT could play a significant role in the development of BPH, compounds were synthesized that could inhibit the conversion of testosterone to its reduced derivative. A 4-azasteroid derivative was found to inhibit the Type I human reductase with a K₁ of 325 nM and the type II enzyme with a K₁ of 12 nM, and the compound, named finasteride, was approved in the United States for the treatment of BPH [Liang, T., Heiss, C., Cheung, A., et al., 4-Azasteroidal 5α-reductase inhibitors without affinity for the androgen receptor. J. Biol. Chem. 259: 734-739, 1984]. Finasteride improves urinary flow rates by shrinking the prostate by 20 to 30 percent and, with long-term use, the drug reduces the need for surgical intervention for BPH from about 10 percent to 5 percent [McConnell, J., Bruskewitz, R., et al., The effect of finasteride on the risk of acute urinary retention and the need for surgical treatment among men with benign prostatic hyperplasia N. Engl. J. Med. 338: 557-563, 1998]. In addition, finasteride was recently shown to provide chemoprevention for prostate cancer. Thus, in a seven-year trial with 18,000 men, the cumulative incidence of cancer was reduced from 24.4 percent in the placebo group to 18.4 percent in the group administered 5 mg of finasteride per day [Thompson, I., Goodman, P., et al., The influence of finasteride on the development of prostate cancer. N. Engl. J. Med. 349: 215-224, 2003].

Other alternative therapies have been used to treat prostate disorders, and a number of clinical trials have been designed to test the effectiveness of various dietary supplementation strategies, such as selenium and α-tocophorol/β-carotene [Revel, C, Method and composition for the treatment of benign prostate hypertophy (BPH) and prevention of prostate cancer, U.S. Pat. No. 6,399,115, Jun. 4, 2002; Heinonen, O., Albanes, D., et al. Prostate cancer and supplementation with α-tocophorol and β-carotene: incidence and mortality in a controlled trial. J. Nat/ Cancer Inst 90: 440-446, 1998; Clark, L., Dalkin, B., et al. Decreased incidence of prostate cancer with selenium supplementation: results of a double-blind cancer prevention trial. Br. J. Urol 81: 730-734, 1998]. The most commonly dispensed phytotherapeutic products contain β-sitosterol, a phytosterol that is found in extracts of Hypoxis rooperi and sold in Europe as Harzol or Azuprostat. Since the biochemical mechanism of action of phytosterols remains unknown and since there may be compositional differences from preparation to preparation, it has been difficult to perform a careful analysis of clinical results using these products [Fagelman, E. & Lowe, F C, Herbal medications in the treatment of benign prostatic hyperplasia (BPH). Urologic Clinics of North America 29: 23-29, 2002]. Thus, there is a prejudice against β-sitosterol as an agent for the treatment and improvement of the symptoms associated with BPH.

This prejudice was confirmed in two clinical trials that compared the efficacy of finasteride with that for a number of nutritional supplements that contained β-sitosterol as the putative active agent. Both studies showed that under the same conditions that produced a 65% reduction in circulating serum DHT by finasteride, there was no effect from products that contained α-sitosterol [Rhodes, L., Primka, R L, Berman, C., et al. “Comparison of finasteride (Proscar), a 5-alpha reductase inhibitor, and various commercial plant extracts in in vitro and in vivo 5 alpha reductase inhibition. Prostate 22: 43-51 (1993); Strauch, G., Perles, P., Vergult, G., et al. “Comparison of finasteride (Proscar) and Serenoa repens (Permixon) in the inhibition of 5-alpha reductase in healthy male volunteers. Eur. Urol. 26: 247-252 (1994)].

Plant-derived sterols (sitosterol, campesterol, stigmasterol, sitostanol, campestanol etc.) are very insoluble in water and in aqueous solutions of bile salt, a major challenge for the delivery of plant sterols as therapeutic agents. This solubility problem has been addressed in their use as cholesterol reduction agents, and two strategies have been successfully devised to circumvent this difficulty. In the first strategy, free sterols and stanols are esterified with rapeseed oil to produce a phytosterol ester derivative that has far greater solubility in oil than its unesterified derivative. These esters can be delivered in soluble form in fatty food products, such as margarine, mayonnaise and salad dressing. Once in the gut, the esters are hydrolyzed by pancreatic cholesterol esterase and the liberated sterol or stanol can then block free cholesterol uptake by the small intestinal cell [Miettinen, T. A., Puska, P., Gylling, et al., Reduction of serum cholesterol with sitostanol-ester margarine in a mildly hypercholesterolemic population. N. Engl. J. Med. 333: 1308, 1995; Weststrate, J. A., & Meijer, G. W. Plant sterol-enriched margarines and reduction of plasma total- and LDL-cholesterol concentrations in normocholesterolemic and mildly hypercholesterolemic subjects. Eur. J. Clin. Nutr. 52: 334, 1998]. In the second strategy, sitostanol is rendered water-soluble and bioavailable by the formation of a complex with a suitable emulsifier such as lecithin or its derivatives. Using this oil-free system, plant-derived stanols were shown to reduce cholesterol absorption by 36.7% and LDL-cholesterol by 14.3% [Ostlund, R E, Sitostanol formulation with emulsifier to reduce cholesterol absorption and method for preparing and use of same. U.S. Pat. No. 6,063,776, May 16, 2000; Spilburg, C A, Goldberg, A C, McGill, J B, et al., Fat-free foods supplemented with soy stanol-lecithin reduce cholesterol absorption and LDL-cholesterol. J. Am. Diet. Assoc. 103: 577-581, 2003].

These two formulation strategies that are designed to enhance efficacy at the level of the small intestinal cell where cholesterol absorption occurs also enhance the absorption of phytosterols themselves. This leads to the unexpected result that these formulation systems may enhance the therapeutic value of phytosterols for conditions that are unrelated to cholesterol metabolism and that require a biologically effective concentration not just at the surface of the small intestinal cell, but in the circulation where they can modify the concentration of androgens, such as DHT. The unanticipated nature of this concept is supported by the vast and detailed toxicological literature that describes the use of plant sterols as cholesterol reduction agents. For example in one published work, healthy male and female human subjects were studied, but the effect of plant sterols on the level of circulating sex hormones was determined only in female subjects. Moreover, testosterone and DHT were not measured in the male subjects, confirming the prejudice that phytosterols have no effect on the metabolism of male androgens [Ayesh, R, Westrate, J A, Drewitt, P N, and Hepburn, P A, Safety evaluation of phytosterol esters. Part 5. Faecal short-chain fatty acid and microflora content, faecal bacterial enzyme activity and serum female sex hormones in healthy normolipidaemic volunteers consuming a controlled diet either with or without a phytosterol ester-enriched margarine. Food & Chem. Tox. 37: 1127-1138 (1999)].

These new formulation systems provide a novel method for maintaining a sufficient level of plant sterols in the circulation to provide interaction with the enzymes involved in testosterone metabolism and thereby to alter the ratio of testostosterone to dihydrotestosterone. Importantly, for the aqueous based sterol/lecithin formulation system, the level of absorption has already been measured using deuterated compounds, providing a physical chemical framework for choosing systems and dosing levels that might be expected to provide the most efficacious results [Ostlund, R E, McGill, J B, Zeng, C-M, et al., Gastrointestinal absorption and plasma kinetics of soy Δ⁵-phytosterols and phytostanols in humans. Am. J. Endocrinol. Metab. 282: E911-E916 (2002)]. For example, absorption of plant sterols, such as sitosterol and campesterol, was about ten times greater than that for their reduced derivatives, sitostanol and campestanol. Based on this result, it might be expected that a smaller dose of native plant sterols would be required for efficacy than that used for plant stanols.

The current invention provides distinct improvements over current and previous methods for delivering plant derived sterols or their fatty acid esters for altering the concentration of circulating dihydrotestosterone. Plant sterols are available from soy as a byproduct of Vitamin E production or from tall oil and, unlike crude or even fractionated extracts of herbs, beans, seeds and grasses, their purity and composition can be characterized by conventional chemical methods, especially gas chromatography. Their recent approval by the Food and Drug Administration as a cholesterol lowering agent in food products such as margarine and salad dressing and their recommended use by the American Heart Association as an initial step in human cholesterol reduction provide evidence of their acknowledged safety. In addition, the other component in the formulation, soy lecithin, has been used for many years as a food additive and it is generally regarded as safe for all food uses. In combination, these two ingredients provide a predictable method to deliver a consistent dose of plant sterol for altering the ratio of testosterone to dihydrotestosterone. Similarly, esters of plant sterols and stanols when dissolved in oil provide another predictable, safe and well-established delivery system for plant-derived sterols.

### SUMMARY OF THE INVENTION

The present invention describes a composition that contains a plant sterol or plant stanol or their fatty acid esters and an emulsifier for treating conditions that are related to elevated dihydrotestosterone. The compositions can be prepared in a dry form for use as a food ingredient, tablet or capsule. Alternatively, the compositions can be dissolved in oil.

This invention describes a method for lowering the concentration of dihydrotestosterone in humans by the administration of properly formulated ingredients commonly found in grains, oils and vegetables. Thus, when plant derived sterols or their fatty acid esters and lecithin are properly formulated, the combination can be used advantageously to treat conditions that are mediated by dihydrotestosterone, such as benign prostatic hyperplasia, prostate cancer, acne and male pattern baldness.

The formulation is prepared by one of two methods. In the first aqueous-based method, phytosterols or their fatty acid esters and lecithin are dissolved in an appropriate organic solvent at a temperature that allows complete dissolution of the two components. After the solvent is removed, the resulting solid is pulverized, added to water and the slurry is homogenized using sonication, homogenization, microfluidization or any other commonly used method. The aqueous dispersion can be used as a food ingredient or dried by lyophilization, spray drying or other convenient method. Alternatively, the powder can be added back to foods or compressed into a tablet or capsule. In the second oil-based method, sterol esters or stanol esters are added to the fat component of certain food products (margarine, salad dressing, etc) or dissolved in oil and incorporated into a capsule following methods described elsewhere [Wester, I., Palmu, T., Miettenen, T., and Gylling, H. Stanol composition and the use thereof. WO 98/06405, 19 Feb. 1998; Van Amorongen, M., Lievense, L., Van Oosten, C. Method of manufacturing an ester mixture. WO 98/01126, 15 Jan., 1998].

The composition enhances the small intestinal absorption and blood concentration of plant-derived sterols and has at least two components (a) an effective amount of food grade emulsifier to improve the intestinal absorption and (b) a plant sterol or stanol or fatty acid esters thereof where the fatty acid ester moiety is derived from a food source oil.

The food grade emulsifier may be a phospholipid such as lecithin or food grade emulsifiers such as monoglycerides, polysorbates, glyceryl monosterates or sodium stearoyl lactylate.

Fatty acid ester moiety may be derived from food grade oils such as rapeseed oil, sunflower seed oil, cottonseed oil. The weight ratio of food grade emulsifier to sterol, stanol or ester thereof is about 0.1 to 10, preferably about 1.

Alternatively, the plant sterols or stanols or fatty acid esters thereof can be formulated in vegetable oils such as soybean oil, canola oil, rapeseed oil, sunflower oil, safflower oil, corn oil, olive oil or the like.

The weight ratio of the sterol, stanol or ester thereof to vegetable oil is limited to the solubility of the stanol, sterol or fatty acid ester thereof in vegetable oil.

Vitamin E may be added as a stabilizer. The vegetable oil based product may be encapsulated into pharmaceutical capsules to provide a pharmaceutical dosage form or added to food products.

A water dispersible solid form of the composition of the sterol, stanol or esters thereof and emulsifier may be formed by (a) dissolving the composition in organic solvents (b) removing the organic solvent at elevated temperature and vacuum pumping to form a solid mass (c) adding water and homogenizing the solid mass and (d) drying the aqueous dispersion, preferably by spray drying. Preferred solvents are ethyl acetate, hexane, heptane, chloroform, dichloromethane, isopropanol. Generally solvent removal process will result in a solid product with less than 1% solvent.

The solid material formed after solvent removal is pulverized and disbursed in water using a Gaulin homogenizer, French press, a sonicator or a microfluidizer. The aqueous dispersion may be dried and drying acids such as maltrin, starch, silicon dioxide or calcium silicate are added to make the powder more flowable and suitable for formulation.

### DETAILED DESCRIPTION OF THE INVENTION

In general, both formulation methods described herein contain a minimum of two components. In the first method, the components are an emulsifier, such as lecithin or its derivatives, and a plant-derived sterol or its fatty acid ester, both of which must be soluble in an organic solvent. In the second method, the components are an ester of a plant sterol or stanol and an oil in which the ester is soluble.

Numerous emulsifiers have been described, but since this application anticipates a pharmaceutical or food application, those compounds that have been approved for human use are deemed most practical. The preferred emulsifier is lecithin derived from egg yolk, soy beans or any of its chemically modified derivatives, such as lysolecithin. While many grades and forms are available, de-oiled lecithin produces the most consistent results. Typical commercially available examples are Ultralec P, Ultralec F and Ultralec G (Archer Daniels Midland, Decatur, Ill.) or Precept 8160, a powdered, enzyme-modified lecithin (Central Soya, Fort Wayne, Ind.).

A variety of sterols and their ester derivatives can be added to lecithin to enhance their aqueous dispersibility in the gut in the presence of bile salts and bile phospholipid. Plant-derived sterols, especially those derived from soy and tall oil, are the preferred choice since they are currently used in a variety of other products. Specifically, this invention comtemplates the use of mixtures including, but not limited to sitosterol, campesterol, stigmasterol and brassicasterol and their corresponding fatty acid esters prepared as described elsewhere (Wester I., et al., “Stanol Composition and the use thereof”, WO 98/06405). The reduced forms of the above-mentioned sterols and their corresponding esters are the less preferred, since their absorption is from five- to ten-fold less than that of their non-reduced counterparts.

The two components are dissolved in a suitable organic solvent, such as chloroform, dichloromethane, ethyl acetate, pentane, hexane and heptane. The choice of solvent is dictated by the solubility of the components, but the preferred solvents are non-chlorinated and since both components are heat stable, heptane is the most preferred solvent because of its high boiling point, which increases their overall solubility. The weight ratio of the emulsifier to sterol in the final mixture can vary from 0.1 to 10.0, with a preferred ratio of 1.0.

After all the components are dissolved at the desired ratio in the appropriate solvent, the liquid is removed at elevated temperature and residual solvent is removed by pumping under vacuum. Alternatively, the solvent can be removed by atomization as described in U.S. Pat. Nos. 4,508,703 and 4,621,023. Water at elevated temperature, preferably between 65° C. and 100° C., is then added. The mixture is vigorously mixed in a suitable mixer to form a milky solution, which is then homogenized with a sonicator, Gaulin dairy homogenizer or a microfluidizer. The water is then removed by spray drying, lyophilization or some other suitable drying method. Before drying, it is helpful, but not necessary, to add a suitable additive such as silicon dioxide or calcium silicate to produce a flowable powder that has more desirable properties for subsequent handling of the powder. This powder can then be added to suitable excipients for preparation of tablets and capsules. The following excipients are useful but not limiting: microcrystalline cellulose, croscarmellose, polyvinylpyrollidone, silicon dioxide, corn starch, magnesium stearate and magnesium silicate.

There are other methods that can be used to prepare tablets. After the components have been mixed at the appropriate ratio in organic solvent, the solvent can be removed as described above. The solid material so prepared can then be compressed at elevated pressure and extruded into a rope. The rope can be cut in segments to form tablets. This method is similar to that described in U.S. Pat. No. 6,312,703, but the importance of pre-mixing the components in organic solvent was not recognized. While this previous patent produces a tablet, the sterol component may not be as freely dispersible in bile salt and phospholipid if it is not pre-mixed in organic solvent. Alternatively, the solid material that results from homogenization and spray drying can be compressed at high pressure and extruded to form a rope that can be cut into tablets.

One of ordinary skill in the art will recognize that the critical step is the intimate mixing of an emulsifier and the sterol and stanol at the appropriate weight ratio to produce a water-dispersible mix. This process can be achieved by other methods, providing the process preserves the chemical stability and the bioavailability of the various components [U.S. Pat. Nos. 5,676,994 and 5,882,713; Warner et al., Use of starch-lipid composites in low-fat ground beef products. Food Technology, 55, 36-41; Knutson et al., Composition and oil-retaining capacity of jet-cooked starch-oil composites. Cereal Chem., 73, 185-188].

A second formulation strategy may also be employed which takes advantage of the greater solubility of sterol esters in oil. Esterification of sterols and stanols is a commercial process that is used by margarine manufacturers to prepare margarines that can lower human LDL-cholesterol. These processes are well known and they have been described in the literature (Practical Handbook of Soybean Processing and Utilization, D. R. Erickson, ed., AOCS Press, Champaign, Ill., Chapter 19). The fatty acid component of these esters is composed typically of but not limited to oleic, linoleic, palmitic linolenic, lauric, myristic and stearic (Westrate, J A and Meijer (1998), Eur. J. of Clin. Nutr. 52, 334). The plant sterol esters can be dissolved in common vegetable oils, such as that from soybean, canola, rapeseed, sunflower, safflower, corn or olive. The plant sterol esters are added at a concentration that produces an effective dose, but that does not exceed the limit of solubility of the ester in the vegetable oil.

### EXAMPLE 1

Equal weights of soy sterols (Archer Daniels Midland) and soy lecithin were mixed in boiling hexane and the solvent was driven off by boiling. After cooling, residual solvent was removed under vacuum. The solid was added to hot water (160° F.), agitated and the hot, milky solution was passed two times through a Gaulin dairy homogenizer operated at 2500-3000 psi. The solution was then spray dried at an inlet temperature of 200° C. and an outlet temperature of 100° C. To enhance the flow characteristics of the sterol/lecithin, the spray dried material was mixed with Aerosil 200 (Degussa Corporation) and Maltodextrin (Grain Processing Corporation) to give a final preparation that contained the following on a gram basis: 1.0 gram sterols, 1.0 gram lecithin, 0.45 gram Maltodextrin and 0.02 gram Aerosil. One part of this powder was added to 17.5 parts of a powdered commercially available, chocolate flavored breakfast drink. Placebo contained lecithin and maltodextrin. For each subject in the clinical study, breakfast powder was added to water and blended such that the active subjects received 1.825 grams of sterols.

### EXAMPLE 2

The effect of the sterol-containing breakfast drink on the circulating level of human DHT was determined in healthy male subjects, between the ages of 20 and 50 years and whose testosterone was between 400 and 1,000 ng/dL. Nineteen subjects completed the study, and the protocol and consent form were approved by an Institutional Review Board. When a fasted subject reported to the clinic, a blood sample was immediately taken and he was assigned either to the placebo or active group. After consumption of the breakfast drink, the subject was served a breakfast consisting of cold cereal, bagel and jam, designed by the dietitian to contain less than 30% of kcal as fat and less than 10% of kcal as saturated fat. Similarly, when the subject returned 4 hours later for his second blood draw, he was served a lunch with these same criteria. The next blood draw occurred 8 hours after sterol dosing and the subject returned fasted for the final blood draw 24 hours post dosing. After completion of the study, blood samples were analyzed for DHT and the baseline value was the mean of values from the initial and 24-hour blood draws. Percent change in DHT was calculated relative to the baseline value and the following results were obtained:

Change in DHT With Treatment Group*

| | Baseline | | |
|---|---|---|---|
| | Value | Percent Change from Baseline | |
| Group | (ng/dL) | 4 Hours Post Dose | 8 Hours Post Dose |
| Active (n = 9) | 54.1 ± 3.9 | −20.7 ± 3.5 | −13.6 ± 5.6 |
| Placebo (n = 11) | 42.8 ± 5.0 | −8.7 ± 3.4 | −14.0 ± 3.9 |
| Difference | — | −12.2 (0.017) | 0.4 (0.96) |
| (p-value) | | | |

*Values are the mean ± sem

These results show that 4 hours after dosing the DHT level was a statistically significant (p=0.017) 12.2% less than that found in the placebo group, consistent with inhibition of 5α-reductase by formulated plant sterols.

1. A composition that enhances the small intestinal absorption and blood concentration of plant-derived sterols that is composed of at least the following two components:
a) effective amount of a food grade emulsifier.
b) a plant-derived sterol or stanol or fatty acid esters thereof in which the fatty acid ester moiety is derived from a food source oil.

2. A composition according to claim 1 wherein the food grade emulsifier is a phospholipid.

3. A composition according to claim 2 wherein the phospholipid is lecithin.

4. A composition of claim 1 wherein the food grade emulsifier is selected from monoglycerides, polysorbates, glyceryl monosterate and sodium stearoyl lactylate.

5. A composition according to claim 1 wherein the fatty acid moiety of the sterol stanol fatty acid esters are derived from rapeseed oil, sunflower seed oil or cottonseed oil.

6. The composition of claim 1 wherein the weight ratio of the food grade emulsifier to sterol or stanol ester thereof is 0.1 to 10.

7. The composition of claim 6 wherein the weight ratio of the good grade emulsifier to sterol or stanol or esters thereof is about 1.

8. The composition of claim 1 further comprising vitamin E as a stabilizer.

9. A method to produce a water-dispersible form of the composition in claim 1 comprising:
a) dissolving the composition of claim 1 in an organic solvent that renders all the components soluble;
b) removing the organic solvent at elevated temperature and removing the residual solvent by vacuum pumping;
c) adding the solid mass to water and homogenizing the mixture; and
d) drying the aqueous dispersion.

10. The method of claim 9 wherein the aqueous dispersion is dried by lyophilization.

11. The method of claim 9 wherein the organic solvent is heated to a temperature that is less than the temperature of decomposition of any of the components.

12. The method of claim 9 wherein the organic solvent is ethyl acetate, hexane, heptane, chloroform, dichloromethane, isopropanol.

13. The method of claim 9 wherein the method for solvent removal produces a solid that contains less than 1.0% solvent.

14. The method of claim 9 wherein the solid formed after solvent removal is pulverized in a mill, grinder or processor to produce a dispersible powder.

15. The method of claim 9 wherein the powder from claim 14 is added with vigorous stirring to water at a temperature that is less than the decomposition temperature of any of the components.

16. The method of claim 9 wherein water is introduced directly into the apparatus that contains the un-pulverized dried solid.

17. The method of claim 16 wherein the water is at a temperature that is less than the decomposition temperature of any of the components.

18. The method of claim 9 wherein the aqueous mixture is homogenized using a Gaulin homogenizer, a French press, a sonicator or a microfluidizer.

19. The method of claim 9 wherein the homogenized aqueous mixture is dried using a spray drier or lyophilizer or other suitable apparatus for the removal of water.

20. The method of claim 9 wherein drying aids, selected from maltrin, starch, silicon dioxide or calcium silicate are added to prevent sticking and to assist in the preparation of a flowable powder.

21. A composition that enhances the small intestinal absorption and blood concentration of plant-derived sterols that is composed of at least the following two components:
a) a plant-derived sterol or stanol or their fatty acid esters in which the fatty acid ester moiety is derived from rapeseed oil, sunflower seed oil, soybean oil or cottonseed oil.
b) a vegetable oil, selected from soybean, canola, rapeseed, sunflower, safflower, corn or olive oil.

22. The composition of claim 21 wherein the fatty acid ester of the plant sterol or stanol is added at a weight ratio to produce an effective dose, but not beyond the limit of solubility of the ester in the oil of choice.

23. The composition of claim 21 wherein vitamin E is added to enhance the stability of the preparation.

24. The composition of claim 21 wherein the sterol and oil is encapsulated in a pharmaceutical capsule.

25. The composition of claim 21 wherein the sterol and oil is added to a food product.

26. The composition of claim 21 wherein the solid is converted into a tablet or capsule as a delivery system for the plant-derived sterol.

27. A solid product that is formed from the composition in claim 21 by subjecting the material to compression or extrusion for at least 15 seconds at a pressure of at least 100 psig.

28. A solid product that is formed from the composition in claim 21 by subjecting the material to compression or extrusion for at least 15 seconds at a pressure of at least 100 psig.
